(19) United States
(12) Patent Application Publication
(10) Pub. No.: US 2003/0205232 A1
(43) Pub. Date: Nov. 6, 2003

(54) Pad for vibration dampening and carpel tunnel syndrome prevention

(76) Inventor: A. Robert Spitzer, Southfield, MI (US)

Correspondence Address: Kenneth I Kohn, Kohn & Associates, Suite 410, 30500 Northwestern Highway, Farmington Hills, MI, 48334, US

(21) Appl. No.: 10/276,945

(22) PCT Filed: Jun. 1, 2001
(86) PCT No.: PCT/US01/17825

(51) Int. Cl. A61F 5/00

(52) U.S. Cl. 128/846; 602/21; 128/879; 128/892

(57) ABSTRACT

A hand pad apparatus (10) for protecting the median nerve, dampening vibration, and absorbing shock including a protecting mechanism (12) for preventing the application of pressure to the median nerve. The protecting mechanism (12) includes parallel cushion portions (24) defining a recess (14) therebetween and a recess maintaining mechanism (22) for maintaining the recess between the cushion portion from reducing the size of the recess. A method of combining median nerve protection, vibration dampening, and shock absorbing to inhibit or prevent carpal tunnel syndrome and a method of determining the proper width of a recess defined by parallel portions of a cushioned pads by determining the type of the cushioned pad material.

### BACKGROUND OF THE INVENTION

1. Field of the Invention

This invention generally relates to the field of grips and pads for the protection of a median nerve from pressure thereon, dampening of vibration and absorption of shock.

2. Description of Related Art

Carpal tunnel syndrome is a common hand condition that occurs by the compression of a median nerve. The transverse carpal ligament forms over the median nerve and compresses it as a result of pressure on the hand, producing symptoms of pain, numbness, weakness, paraesthesia, or atrophy in the territory of the median nerve distal to the course through the carpal tunnel in the wrist. Traumatic injury is exacerbated because of the narrowness of the carpal canal. There is no opportunity for the nerve to be displaced away from any compressive forces. Additionally, compressive injury is exacerbated by the development of edema in the tissues within the canal, which, because of the relatively fixed size, causes added injury due to compression of the nerve within the canal. An additional component of the injury is associated with friction between the nerve and the adjacent tendons caused by repetitive motions that are worsened by compression and further reduce the available space within the canal.

Carpal tunnel syndrome is a particular problem for workers in industries that require manual operations with hand held implements or tools or in office situations wherein a worker is required to spend several hours a day resting their hands on a typewriter or computer terminal. Carpal tunnel syndrome also occurs due to sports related activities.

Two types of injury can occur due to compression of the median nerve. The milder, earlier form is a demyelination of the median nerve within the canal. This type of demyelination occurs early in the course of compressive injury. This form of injury however, is more readily reversible and recovery occurs in four to six weeks after compression is relieved. This form of injury can cause motor weakness due to acute conduction block in the nerve; however, this weakness is readily reversible.

The second major form of injury includes damage to the axons themselves. This form of injury occurs in more severe or prolonged cases and has more significant implications in that it often leads to motor weakness. This type of motor weakness tends to be poorly reversible and often irreversible. Loss of strength in the thenar muscles can lead to major disabilities due to the loss of ability to grip or perform fine dexterous manipulations. This second form of injury is generally seen in long-standing cases, which first clinically manifest in a manner suggestive of the demyelinating form.

If the median nerve is injured at the wrist, as by wounds or by a dislocation of the lunate bone, sensation is usually lost in the skin on the front of the index finger and adjacent part of the thumb and over the back of the distal phalanges of the thumb, index finger, and middle fingers, and is diminished over a large area. The brunt of the paralysis falls on the muscles of the thenar eminence which, in time, flattens and wastes.

Treatment of carpal tunnel syndrome varies according to the severity of the condition. Severe conditions usually require hand surgery to sever the transverse carpal ligament, whereas in less severe cases, a splint may be utilized to immobilize the wrist.

In order to prevent or inhibit a person from development carpal tunnel syndrome, a number of gloves and wrist braces have been designed. One such glove is disclosed in U.S. Pat. No. 4,850,341 to Fabry et al., issued Jul. 25, 1989, which discloses a glove for inhibiting or preventing carpal tunnel syndrome, which includes a pad configured to cover and protect the median nerve of the wearer's hand. The problem with such a device is that the pad is placed directly over the median nerve and, therefore, transmits pressure from the external source to the carpal tunnel ligament. With this type of relationship, repetitive finger movements can increase the potential for frictional injury. Additionally, a pad oriented directly over the median nerve allows for the direct transmission of pressure to the median nerve.

In order to overcome the problems associated with the type of glove disclosed in the Fabry et al. patent, a pad design was disclosed in U.S. Pat. No. 5,031,640 to Applicant, issued Jul. 16, 1991 and incorporated herein by reference, which eliminates the continuous pressure applied directly over the median nerve by providing a recess over the median nerve in a support pad, thereby preventing or eliminating carpal tunnel syndrome. This type of design has proven to be very effective in inhibiting or preventing carpal tunnel syndrome.

Additionally, U.S. Pat. No. 6,006,751 to Applicant discloses a glove for preventing carpal tunnel syndrome. The glove assembly is adapted to inhibit or prevent carpal tunnel syndrome. The glove includes a flexible glove body having a front side and a back side that defines a wrist opening and at least one finger opening. The glove further includes a resilient protector secured to the front side of the glove body for preventing the application of pressure to the median nerve. The resilient protector defines a recess extending substantially parallel with both sides of the median nerve.

In addition to the above-described patents, there are many different types of handgrips available that provide a pad for comfort. These pads mainly prevent external vibration and shock from causing injury to the hands and are normally made of a continuous rubber material including grooves that are decorative in nature. Moreover, by their nature, these pads are soft and compressible in order to absorb vibration. More softness means more vibration dampening, but easy compression of the pads.

The currently existing pads themselves do not provide any protection to the median nerve. In fact, there are no pads that eliminate continuous pressure directly over the median nerve and thereby prevent carpal tunnel syndrome, while also dampening vibration and absorbing-shock. The use of a soft pad material for vibration damping and absorption is considered in conflict with the need for a non-compressible pad to prevent pressure from being applied to the median nerve. In combination with the previously described patents, a soft vibration damping material fails to protect the median nerve when the material is compressed by an external pressure source onto the palm of the hand. The compressed pad material expands laterally, such that it closes a significant portion of the recess defined in the previously disclosed inventions. Therefore, pressure is transmitted into the recess and the median nerve is not protected. If the pad is compressed sufficiently and in combination with some deformation of the palm itself, the surface of the external pressure is applied to the palm and the protection is lost. This occurs under static load, or transiently during impact or vibratory oscillation.

Accordingly, there is a need for a device that protects the median nerve from external pressure and external vibration and shock.

### SUMMARY OF THE INVENTION

According to the present invention, there is provided a hand pad apparatus for protecting a median nerve, dampening vibration, and absorbing shock including a protecting mechanism for preventing the application of pressure to the median nerve. The protecting mechanism includes parallel cushion portions defining a recess therebetween and a recess maintaining mechanism for maintaining the recess between the cushion portions. The recess maintaining mechanism prevents the cushion portions from reducing the size of the recess. Additionally, the present invention provides for a protecting mechanism including a recess that does not reduce in size after the cushion portions of the protecting mechanism are compressed. Further, the present invention provides for a method of combining median nerve protection, vibration dampening, and shock absorbing to inhibit or prevent carpal tunnel syndrome including the steps of providing the hand pad apparatus and disposing the hand pad apparatus onto a hand, while maintaining the recess over the median nerve. Finally, the present invention includes a method of determining the proper width of a recess defined by parallel portions of a cushioned pad including the steps of determining the material type of the cushioned pad and its hardness; and empirically calculating the proper thickness of the cushioned pad to maintain the recess at a width at least as wide as a median nerve.

### DESCRIPTION OF THE DRAWINGS

Other advantages of the present invention will be readily appreciated as the same becomes better understood by reference to the following detailed description when considered in connection with the accompanying drawings wherein:

FIG. 1 is a top view of an embodiment of the present invention made of a protecting mechanism including parallel cushion portions defining a recess extending therebetween and a recess maintaining mechanism for maintaining the recess between the cushion portions, thereby preventing the cushion portions from reducing the size of the recess;

FIG. 2 is a cross-sectional perspective view of an embodiment of the present invention made of the protecting mechanism, cushion portions, recess and recess maintaining mechanism;

FIG. 3 is a top view of an embodiment of the present invention being oriented onto the palm of a hand and having a varied recess;

FIG. 4 is an embodiment of the present invention wherein the hand pad apparatus is disposed within a glove; and

FIG. 5 is an embodiment of the present invention wherein the hand pad apparatus is composed of numerous individual cushion portions and disposed within a glove.

### DETAILED DESCRIPTION OF THE INVENTION

The present invention provides for a hand pad apparatus, generally shown at 10 in the Figures, for inhibiting or preventing carpal tunnel syndrome and for dampening vibration and absorbing shock. Generally, the hand pad apparatus 10 is composed of a protecting mechanism 12 having a configured recess 14 placed within the protecting mechanism 12 itself. The recess 14 is located and placed over and co-axial with a median nerve 16 of a hand 18. The recess 14 is substantially parallel with the median nerve 16. In an embodiment of the present invention, the hand pad apparatus 10 is designed solely for the placement directly onto the palm of the hand 18.

The present invention is applicable for use in any setting including, but not limited to, offices, factories, homes, industries, businesses, sporting events, recreational activities, and any other similar settings requiring the use of hands being in direct contact with tools, instruments, and devices. The hand pad apparatus 10 is well suited in uses dealing with the transmission of mechanical energy such as shock or vibration. Such mechanical energy originates from sources including, but not limited to, mechanical tools, metal bars, and sporting apparatus. Thus, individuals who have pressure applied directly to their hands greatly benefit from the hand pad apparatus 10 disclosed herein.

The protecting mechanism 12 of the hand pad apparatus 10 is a cushioned pad 24 comprising vibration damping and shock absorbing material or compounds including parallel cushion portions 24. The parallel cushion portions 24 are composed of materials including, but not limited to, elastomeric material, vulcanized elastomeric material, cis polybutadiene rubber, styrene butadiene rubber, rubber, neoprene, ehtylene propylene terpolymer, styrene butadiene, polyethelene, urethane, air, foam, silicone gel, and any other similar vibration dampening and shock absorbing materials known to those of skill in the art. Generally, these materials or compounds have good abrasion, corrosion, and impact resistance properties. Furthermore, open or closed celled materials are adaptable for use in the present invention. Additionally, if desired, the pad materials are cut to have a waffle pattern. Finally, the cushioned pads 24 are lightweight, malleable, and easily cut to be fabricated into various shapes, contours, and sizes in order to be useful in a wide array of applications.

The critical characteristic of the hand pad apparatus 10 is that it protects the median nerve 16 while dampening vibration and absorbing shock. The recess 14 included in the protecting mechanism 12 prevents the application of pressure to the median nerve 16. The pressure instead is distributed to the cushion portions 24 of the protecting mechanism 12 and other areas of the protecting mechanism 12. However, as pressure is applied to the hand pad apparatus 10, the protecting mechanism 12 is compressed. The compression causes lateral expansion and spread of the cushion portions 24 of the protecting mechanism 12. The expansion is generally uniform so that expansion of the cushion portion 24 of the protecting mechanism 12 into the recess 14 occurs. Thus, the size of the recess 14 is reduced.

The degree of expansion and spreading of the cushion portions 24 of the protecting mechanism 12 varies depending upon the hardness of the materials or compounds composing the protecting mechanism 12. In applications where vibration absorption is required, materials or compounds that have a high degree of softness is used. As a result, a high degree of lateral spreading, especially into the recess 14 occurs when pressure is applied to the protecting mechanism 12. Once the recess 14 is reduced in size due to the lateral expansion and spreading of the cushion portions 24 of the protecting mechanism 12, the opposing, parallel cushion portions 24 of the protecting mechanism 12 narrow to a point of contact with the median nerve 16. Thus, protection of the median nerve 16 does not occur.

The present invention resolves the problem associated with the lateral expansion and spread of the cushion portions 24 of the protecting mechanism 12 by either employing a recess maintaining mechanism 22 or by varying the size of recess depending upon the type, hardness, and thickness of the pad material. In either embodiment, the cushion portion 24 of the protecting mechanism 12 does not significantly expand into the recess 14 and thus the size of the recess 14 is not reduced and protection of the median nerve 16 occurs.

In the embodiment of the present invention utilizing the recess maintaining mechanism 22, the recess maintaining mechanism 22 can be various mechanisms that prevents the recess 14 from being reduced in size so as to allow protection of the median nerve 16. Preferably, the recess maintaining mechanism 22 contains a layer of rigid material disposed around the recess 14. The rigid material includes, but is not limited to, plastic, harden rubber, metal, wood, cloth, thread, the cushion portion 24 itself, and any other similar material that prevents the reduction of the size of the recess 14 over the median nerve 16. The recess maintaining means 22 prevents the lateral expansion of the cushion portion 24 into the recess 14 when pressure is applied to the protecting mechanism 12. Expansion of the cushion 24 is forced outwardly therefrom, but inward expansion into the recess 14 is lessened sufficiently and/or totally prevented.

In another embodiment of the present invention, the recess 14 is varied in size depending upon the type, hardness, and thickness of the cushion pad 24 material. Alternatively, the thickness of the pad material is varied according to the type, hardness and width of the recess 14 between the parallel cushion portions 24 of the protecting mechanism 12. The softness or hardness of the pad material is key in determining the proper dimensions and configuration of the apparatus 10. Additionally, the amount of applied static and transient loads being applied to the hand pad apparatus 10 is an influential factor. The proper width of the recess 14 and/or thickness of the cushion portions 24 of the protecting mechanism 12 will vary in order to prevent any pressure from being applied to the median nerve 16.

The appropriate combination of width of the recess 14 and thickness of the pad material is empirically determined for a specific application. The first step in determining the appropriate combination is to determine the specific application, which involves determining the static load pressure and desired degree of vibration damping. Once these factors are determined, an appropriate material is selected according to its hardness or softness. The hardness or softness of the pad material is determined through the use of a durometer or through any method known to those of skill in the art. Generally, a compromise on hardness is necessary to prevent extreme compression of the pad material and subsequent loss of median nerve 16 protection.

After choosing the appropriate pad material, the pad material must be configured to fit various sized hands. Then, the width of the recess 14 and the thickness of the pad material are adjusted to prevent the recess 14 between the parallel cushion portions 24 of the protecting mechanism 12 from becoming narrower than the width of the median nerve 16 at the maximum static pressure, impact pressure, or vibration. The greatest value of the static pressure, impact pressure or vibration is used to ultimately determine the width of the recess 14 and desired thickness of the pad material. In some situations however, rare extreme transient compression is tolerable. Generally speaking, for a soft material, vibration damping is enhanced, but the pad thickness is greater. For materials with a great deal of lateral expansion and spread under pressure, the recess 14 needs to be wider. For those with minimal lateral expansion and spread, the recess 14 is narrowed.

The apparatus is adaptable for use by itself as a hand grip (FIG. 3) or it is easily adapted to be inserted into a palm surface of a glove assembly 20 to retain its position on the palm of the hand 18 and to maintain the recess 14 over the median nerve 16. Generally, the glove assembly 20 is a flexible glove body including a front and back side and at least one bridging mechanism 26 disposed between the cushion portions 24 of the protecting mechanism 12 and over the recess 14. The glove assembly 20 is constructed of a flexible or expandable elastic-type material that conforms to the hand 18 of the user. The flexible material provides a more secure fit for the glove assembly 20. The flexible material is a fabric that imparts flexibility to the glove assembly 20, wherein such fabric includes SPANDEX or other similar fabric. However, the glove assembly 20 is made of any material including, but not limited to, cotton, wool, leather, and synthetic materials such as nylons (Kevlar™ of DuPont) and polyesters, combinations thereof, and any other similar material known to those of skill in the art.

The bridging mechanism 26 of the glove assembly 20 is constructed of a material that is less resilient than the material that comprises the cushion portions 24 of the protecting mechanism 12. The bridging mechanism 26 has many forms and shapes necessary to prevent the penetration or infiltration of external objects into the recess 14, but preferably is “domed” shaped and extends away from the surface of the cushion portion 24. The glove assembly 20 includes at least one bridge 26, but usually includes a number of bridges 26 in order to provide adequate coverage of the recess 14 and protection of the median nerve 16. The bridge 26 is connected to the cushion portions 24 through an elastic portion. The bridge 26 is constructed from any suitable material known to those of skill in the art including, but not limited to, plastic materials such as polystyrene, polyvinyl chloride, polyurethane, as well as metals, utilizing methods known to those of skill in the art of plastic injection, molding and forming.

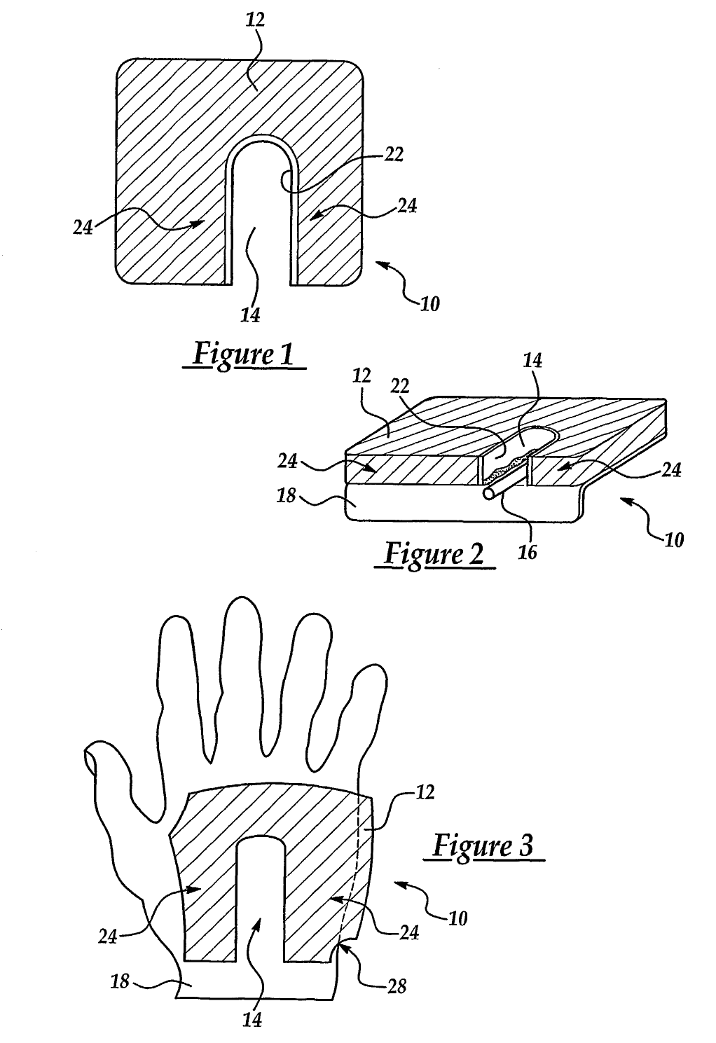

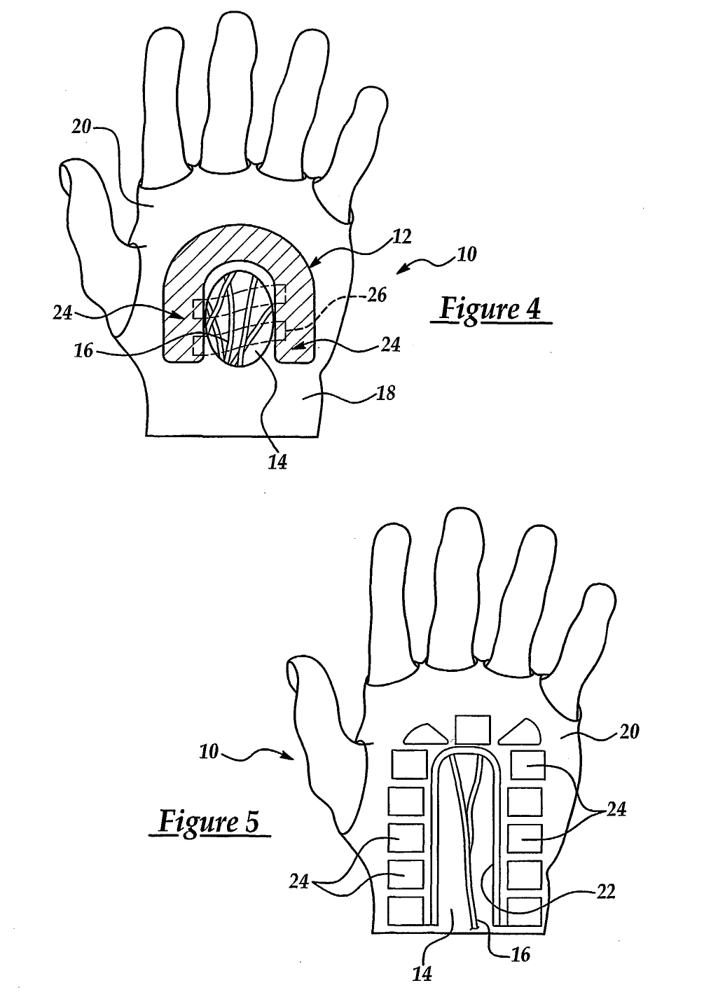

As best illustrated in FIG. 4, the protecting mechanism 12 is secured to the palm side of the glove assembly 20. The protecting mechanism 12 has numerous configurations including, but not limited to, several individual cushions 14 or pads 14 (FIG. 5), a single, cushion 14 or pad 14 (FIGS. 1 and 2), a horseshoe shaped pad 14 (FIG. 4) having recessed portions in the surface thereof to provide flex areas 28 between the relatively raised portions, and any configuration of the cushion 14 or pad 14 being affixed to the glove assembly 20.

In operation, the present invention includes the steps of providing the hand pad apparatus 10 including the protecting mechanism 12, parallel cushion portions 24, the recess 14, and the recess maintaining mechanism 22; and disposing the hand pad apparatus 10 on a hand 18 while maintaining the position of the recess 14 over the median nerve 16. Additionally, the method further includes disposing the apparatus 10 within a glove assembly 20 to be placed onto the person's hand 18.

### EXAMPLE

For specific applications exact parameters of pad material hardness, recess 14 width, and pad material thickness is determined empirically. For use in the industrial work glove environment, experiments have shown success in combining the properties of median nerve protection and vibrations using the following typical values:

(1) recess width typically 5 mm to 15 mm;

(2) pad material hardness of Shor A 14-20; and

(3) pad thickness of 150 mm to 250 mm.

These values are meant as typical examples for a range of industrial and sporting applications, but are not meant to represent every possible combination of values that are used. These values were determined primarily with urethane compounds, but other pad materials, such as air filled pads and others described herein, result in different optimal combined values.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

What is claimed is:

1. A hand pad apparatus for protecting a median nerve, dampening vibration, and absorbing shock comprising protecting means for preventing the application of pressure to the median nerve, said protecting means including parallel cushion portions defining a recess therebetween and recess maintaining means for maintaining said recess between said cushion portions, thereby preventing said cushion portions from reducing the size of said recess.

2. The hand pad apparatus according to claim 1, wherein said protecting means is at least one cushion pad made of vibration absorbing material selected from the group consisting essentially of elastomeric material, urethane, air, foam, polyethylene, rubber, and silicone gel.

3. The hand pad apparatus according to claim 2, wherein said elastomeric material is selected from the group consisting essentially of neoprene, ethylene propylene terpolymer and styrene butadiene.

4. The hand pad apparatus according to claim 2, wherein said rubber is made from materials selected from the group consisting essentially of cis polybutadiene rubber, styrene butadiene rubber, and natural rubber.

5. The hand pad apparatus according to claim 1, wherein said recess maintaining means is defined as material disposed about said protecting means proximate to said recess that prevents lateral expansion of the protecting means into said recess.

6. The hand pad apparatus according to claim 5, wherein said material is made of material selected from the group consisting essentially of plastic, harden rubber, metal, wood, cushion pad, and thread.

7. The hand pad apparatus according to claim 1, wherein said recess is at least as wide as the median nerve and remains at least as wide as the median nerve after pressure is applied to said apparatus.

8. The hand pad apparatus according to claim 1, further defined as a hand grip assembly for placement between a hand and a tool.

9. The hand pad apparatus according to claim 1, wherein said protecting means is disposed within a glove assembly.

10. The hand pad apparatus according to claim 9, wherein said glove assembly comprises a flexible glove body including a front side and a back side and at least one bridging means disposed between said cushion portions of said protecting means and over said recess.

11. The hand pad apparatus according to claim 10, wherein said bridging means is made of a plastic material including polystyrene, polyvinyl chloride, and polyurethane.

12. The hand pad apparatus according to claim 10, wherein said bridging means is affixed to said cushion portions of said protecting means.

13. A hand pad apparatus for protecting a median nerve, dampening vibration, and absorbing shock comprising protecting means for preventing the application of pressure to the median nerve, said protecting means including parallel cushion portions defining a recess therebetween wherein said recess remains open over the median nerve.

14. The hand pad apparatus of claim 13, wherein said protecting means is at least one cushioned pad made of vibration absorbing material selected from the group consisting essentially of elastomeric material, urethane, air, foam, polyethylene, rubber, and silicone gel.

15. The hand pad apparatus according to claim 14, wherein said elastomeric material is selected from the group consisting essentially of neoprene, ethylene propylene terpolymer and styrene butadiene.

16. The hand pad apparatus according to claim 14, wherein said rubber is made from materials selected from the group consisting essentially of cis polybutadiene rubber, styrene butadiene rubber, and natural rubber.

17. The hand pad apparatus according to claim 13, further defined as a hand grip assembly for placement between a hand and a tool.

18. The hand pad apparatus according to claim 13, wherein said protecting means is disposed within a glove assembly.

19. The hand pad apparatus according to claim 18, wherein said glove assembly comprises a flexible glove body including a front side and a back side and at least one bridging means disposed between said cushion portions of said protecting means and over said recess.

20. The hand pad apparatus according to claim 18, wherein said bridging means is made of a plastic material including polystyrene, polyvinyl chloride, and polyurethane.

21. The hand pad apparatus according to claim 20, wherein said bridging means is affixed to said cushion portions of said protecting means.

22. The hand pad apparatus according to claim 13, wherein said recess remains open as wide as the median nerve after said cushion portions are compressed thereon.

23. A method of combining median nerve protection, vibration dampening, and shock absorbing to inhibit and prevent carpal tunnel syndrome comprising the steps of:
preventing the application of pressure to the median nerve by disposing thereabout parallel cushion portions defining a recess therebetween, the recess being substantially parallel with both sides of the median nerve; and
maintaining the recess about the median nerve with pressure.

24. The method according to claim 23, wherein said disposing step includes disposing the apparatus within a glove to be placed on the person's hand.

25. A method of determining the proper width of a recess defined by parallel portions of a pad material, wherein the steps comprise:
determining the material type of the cushioned pad and its hardness; and
empirically calculating the proper thickness of the material to maintain the recess at a width at least as wide as a median nerve as a maximum amount of pressure is applied to the pad material thereon.

26. The method according to claim 25, wherein said determining step is further defined as utilizing a durometer to determine the hardness of the cushioned pad.

27. The method according to claim 25, wherein said calculating step includes using pressure originating from the group consisting essentially of static loads, static pressure, transient pressure, and vibration.

28. A method of determining the proper thickness of pad material used as protecting means for preventing the application of pressure to a median nerve, dampening vibration, and absorbing shock, comprising the steps of:
selecting the pad material;
determining the hardness of the pad material;
fabricating parallel portions of the pad material to define a recess therebetween, wherein the recess is substantially parallel with both sides of the median nerve; and
empirically calculating the proper thickness of the material to keep the recess opening at least as wide as the median nerve when maximum pressure is applied to the pad material thereon.
